(19) United States
(12) Patent Application Publication
(10) Pub. No.: US 2009/0260127 A1
(43) Pub. Date: Oct. 22, 2009

(54) Garment

(76) Inventor: Cynthia Marie Prezgar, Falls Church, VA (US)

Correspondence Address: Cynthia Prezgar, Suite 800, 1390 Chain Bridge Road, McLean, VA, 22101, US

(21) Appl. No.: 12/384,877

(22) Filed: Apr. 9, 2009

Related U.S. Application Data
(60) Provisional application No. 61/124,060, filed on Apr. 14, 2008.

(51) Int. Cl. A41D 3/08; A41B 3/00; A41D 27/20; A61F 7/00

(52) U.S. Cl. 2/88; 2/129; 2/250; 607/109; 607/112

(57) ABSTRACT

The improved garment is constructed in a circular and attached manner effectively providing superior functionality, warmth, water repellence, convenience, comfort, ease of use and fashion. Further, particularly for those with physical limitations, the garment provides a higher degree of safety than a piece of clothing that can easily slip out of place or fall to the ground. With the garment hanging around the shoulders or neck, the wearer can position the twisted area and the garment to allow multiple versatile and useful alternatives. The improved garment is unique in shape and composition and provides added functionality, warmth, water repellence, convenience, and comfort for the wearer in a manner far superior to traditional shawls, scarves, sweaters and headwear. The garment can be constructed in such a way that each side of the product is of a varying fabric, color, or other quality thus providing enhanced usefulness, versatility and cost effectiveness.

### REFERENCE TO RELATED APPLICATIONS

This application is entitled to the benefits of Provisional Patent Application Ser. No. 61/124,060 filed 2008 Apr. 14.

### STATEMENT REGARDING FEDERALLY SPONSORED RESEARCH OR DEVELOPMENT

Not Applicable

### REFERENCE TO SEQUENCE LISTING

Not applicable

### COPYRIGHT NOTICE

A portion of the disclosure of this patent document contains or may contain material which is subject to copyright protection. The copyright owner has not objection to the photocopy reproduction by anyone of the patent document or the patent disclosure in exactly the form it appears in the Patent and Trademark Office patent file or records, but otherwise reserves all copyright rights whatsoever.

### BACKGROUND OF THE INVENTION

The present invention relates to the field of garments, shawls, scarves, outerwear, headwear, neckwear, and the like. More specifically, this invention relates to outerwear that can take the form of a shawl, poncho, scarf or hood, and also can exist as both shawl and hood or scarf and hood or shawl and scarf simultaneously. Uniquely, this invention applies to the field of cost effective convertible outerwear.

Traditional shawls and scarves have the drawback of slipping. The wearer is inconvenienced by clothing that does not effectively, easily or comfortably stay in place.

In Adjustable Scarf U.S. Pat. No. 3,748,661 the resulting product provides a more secure scarf however, the functionality is limited.

In Shawl U.S. Pat. No. 7,222,371 the shawl includes closures which while providing advantages, limits functionality and basically provides a single valuable use, that of adding warmth for a person with physical limitations.

In Garment U.S. Pat. No. 6,119,270 the product includes sleeves and zippers to allow the garment to be easily taken on and off for a variety of wearers including patients. However, the sleeves of this product could result in discomfort for the patient. Also, manipulating the sleeves and zippers could be very time consuming for the caregiver.

In Scarf with a Twist U.S. Pat. No. 5,867,835 the product is essentially worn around the neck as a fashion item. As such, the functionality is limited.

In Scarf with Knot Pleat U.S. Pat. No. 6,065,156 the product is a securely fitting scarf. However, the functionality is limited to that of a scarf only.

In Garment U.S. Pat. No. 4,060,853 the product forms its own carrying case thereby making it easy to transport the jacket when it is not being worn. However, it is still necessary for the wearer to hold the carry case when the jacket it not in use.

In Shawl U.S. Pat. No. 1,157,341 the product includes a strip of material secured at each wrist. While this allows for a single alternative of secure positioning it is limited in functionality.

In Shawl Type Garment U.S. Pat. No. 6,240,562 the product simulates a sweater worn as a shawl or mantle. However, the functionality is limited to this one alternative.

In Multiple Purpose Scarf U.S. Pat. No. 4,870,707 the product can be adapted for use as different articles of clothing. However, this product includes ties which can be inconvenient for the wearer. Additionally, this product in most alternatives does not provide warmth.

In Scarf U.S. Pat. No. 6,226,799 the scarf is constructed such that it can cover the neck, chest, and back without a knot. However, this product requires the use of Velcro, hooks, or some other means of fastening which can be cumbersome for the wearer.

In Warming Scarf U.S. Pat. No. 7,275,269 the scarf effectively covers the neck and some of the chest area. While this product is useful it is also limited in functionality.

In Combination Scarf U.S. Pat. No. 4,404,688 the product can be worn in various formats as ornamentation. However, the functionality is limited to that of a traditional scarf.

In Neck and Chest Scarf U.S. Pat. No. 5,802,618 the product effectively covers the neck chest and face. However, it does not provide warmth or covering for shoulders.

In Nursing Shawl U.S. Pat. No. 4,797,953 the product serves as an effective shawl for a nursing mother. However, the functionality is limited to this single purpose.

In Nursing Cover and Method of Making Same U.S. Pat. No. 5,440,762 the product serves as an effective shawl for a nursing mother. However, functionality is limited to single purpose.

The mobius shawl is by definition one-sided, has one edge and is seamless. While it can provide some varying functionality the construction procedure is complex and the steps which allow the gradual curve creates relative bulk which results in limited ultimate alternative manners of wear. Typical hand crafted items with an alternative mobius format are also limited in functionality.

Therefore, there is a need for an improved garment that will allow varying functionality while effectively staying in place.

### BRIEF SUMMARY OF THE INVENTION

The principal object of the invention described below is to provide a new and improved garment, shawl, scarf, or headwear (or more than one, simultaneously). The invention, in its best mode is embodied as an endless loop of fabric constructed of any of a vast variety of material or substances, designed and assembled such that varying and numerous purposes can be deployed by the user.

Further the unique construction solves the problem of slipping experienced with traditional shawls providing ease of use while enabling varying functionality. In each embodiment the product stays in place providing benefits of comfort and convenience. Additionally, this advantage can minimize or eliminate safety issues resulting from loose or unstable clothing. This is particularly significant when the wearer is sick or elderly. Also, this benefit provides a distinct advantage for nursing mothers.

Additionally, the versatility of the invention allows the single product to be utilized effectively in differing embodiments during distinct seasons and weather conditions thereby resulting in a substantially cost effective garment.

Another object of the instant invention is to provide a piece of outerwear, neckwear, or headwear that will optimally or alternatively present an outer, decorative and visually soft fabric, or an outer weatherproof, water-repellent material.

Still another object of the present invention is to allow either, both sides, or a portion of either or both sides thereof to have individual characteristics.

### BRIEF DESCRIPTION OF THE SEVERAL VIEWS OF THE DRAWINGS

Referring now to the accompanying drawings, constituting a part hereof, it is seen that:

FIG. 1 illustrates in a perspective view the general configuration of the improved garment uniquely constructed in accordance with the present invention.

FIG. 2 presents another view of the present invention positioned in an alternative manner to demonstrate an additional application for the wearer.

FIG. 3 is yet another view of the present invention showing an additional configuration of the inventive product.

FIG. 4 is another view of the inventive product as an example of one mode of application to the human body.

FIG. 5 illustrates a perspective view of an alternative embodiment of the versatile invention.

FIG. 6 illustrates an additional embodiment of the present invention.

FIG. 7 illustrates yet another embodiment of the present invention.

FIG. 8 illustrates an additional embodiment of the present invention.

FIG. 9 is a view of the back of the present invention illustrating example construction details.

FIG. 10 is a view of one version of the invention prior to fold and assembly.

FIG. 11 is a view of the invention with a fold.

FIG. 12 is a view of the invention with a fold with edges positioned prior to assembly.

### DETAILED DESCRIPTION OF THE INVENTION

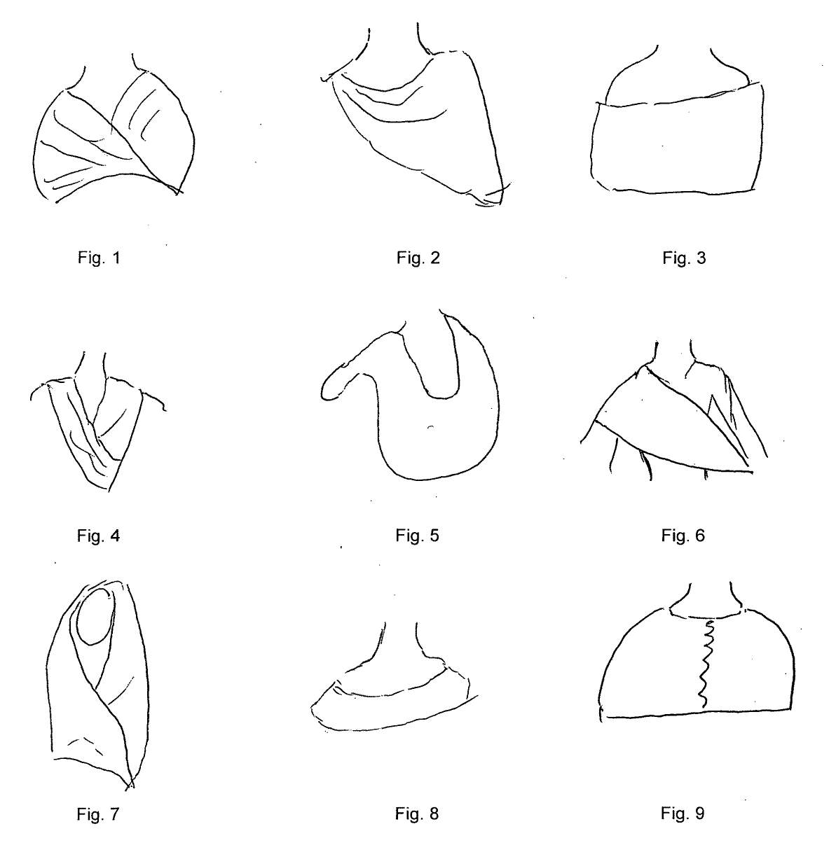

Illustrated in FIG. 1 is a view of the improved garment constructed in accordance with the invention. The garment can be circular in form with a half twist. This half twist is accomplished by folding the material prior to securing a back seam or establishing other means of attachment. The garment is essentially constructed in the format of a mobius strip with modifications enabling varying functionality while staying in place.

The dimension of the improved garment is relational to the circumference of the wearer's shoulders depends on intended use and material deployed. It is constructed of any fabric or raw material including but not limited to knitted or otherwise woven material.

A sample dimension of the improved garment is twenty-six (26) inches high and fifty-four (54) inches along the bottom edge of the shawl and (44) inches along the top edge. However, the dimension of the invention varies based on the design of the panel or panels, intended use, and material deployed.

The improved garment in many possible alternatives can be constructed of a piece or pieces of material cut and assembled in essence in a trapezoid format thereby resulting in the finished garment having a tapered shape resembling an inverted cone. Trapezoid is defined as a quadrilateral having two parallel sides. The edges and corners of the piece of material can be tapered to further enable the attachment, flow and draping which enhances functionality in multiple varying uses.

The invention can serve as a shawl, a scarf or hood and can also exist as both shawl and hood or scarf and hood simultaneously.

In an alternative embodiment the invention can be constructed of a panel or a series of panels connected to form an endless loop such that the resulting garment is essentially a connected shawl with a V shaped neck, scooped neck, or draped neck. As illustrated in FIG. 1 the invention has a tapered shape and resembles an inverted cone.

In this alternative embodiment the V neck, scooped neck, or draped neck results from a series of panels joined accordingly to in essence simulate the appearance and some functionality resulting from the half-twist. The simplicity of this embodiment can be beneficial for elderly individuals for whom the invention minimizes the discomfort often experienced in dressing.

The invention provides distinct advantages for elderly individuals and those with physical limitations. This embodiment provides the advantage of the invention staying in place while the flexible and open neckline allows for less discomfort during dressing and undressing. Also, the relatively large neckline minimizes the fear that might be experienced by, for example, an Alzheimer's patient when having something placed over their head. Further, this embodiment does not interfere with intravenous tubes nor will it slip into the wheels of a wheelchair or fall onto the floor. Additionally, it leaves the wearer's hands free to perform necessary tasks. Various other features can be incorporated including pockets, slits for arms, a collar, as well as other attributes.

Further, the dimensional and construction features are recited as examples only for illustration and are not to be considered as limiting the scope of this invention. Clearly, the invention can take a variety of shapes and forms while remaining well within the scope of coverage of the depending claims.

FIG. 2 illustrates the twist area of the product positioned on the shoulder thereby allowing an alternative comfortable fashionable manner of wear without the problem of slipping typically experienced with traditional shawls.

FIG. 3 illustrates the product worn with the twist area shifted to the wearers back. This delivers an alternative use whereby the product resembles a poncho.

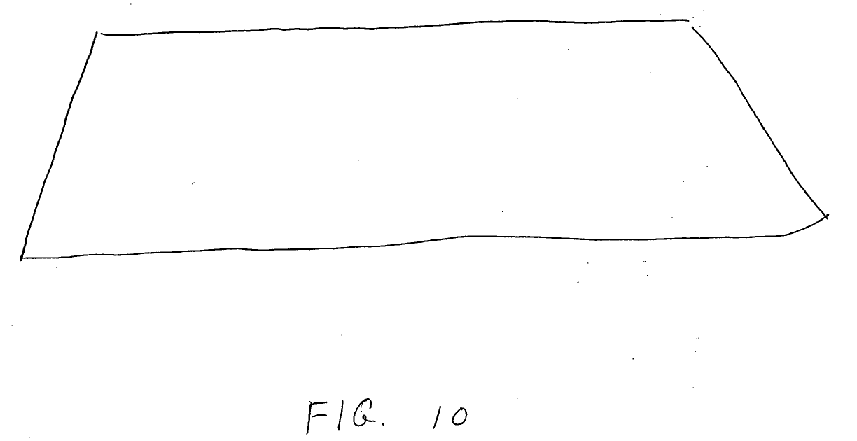

FIG. 4 is a view of the product worn for a variety of purposes including as an alternative to a traditional scarf positioned for fashion or warmth possibly worn under a coat. However, the invention is more secure than a traditional scarf and can be positioned more comfortably and securely around the neck. If worn in cold weather under a coat the product can provide a barrier to cold air in a manner superior to a traditional scarf. Therefore the invention can provide more warmth and comfort than a traditional scarf.

FIG. 5 illustrates the invention worn in an alternative manner. In this view the wearer conveniently positions the product around the neck with most of the shawl flowing down the back. This scenario enables the wearer to effortlessly and securely position the invention if the weather is too warm to comfortably wear the item around the shoulder. In essence, a superior alternative to a traditional sweater tied around the wearer's neck.

FIG. 6 is a view of the product worn in an alternative fashionable manner.

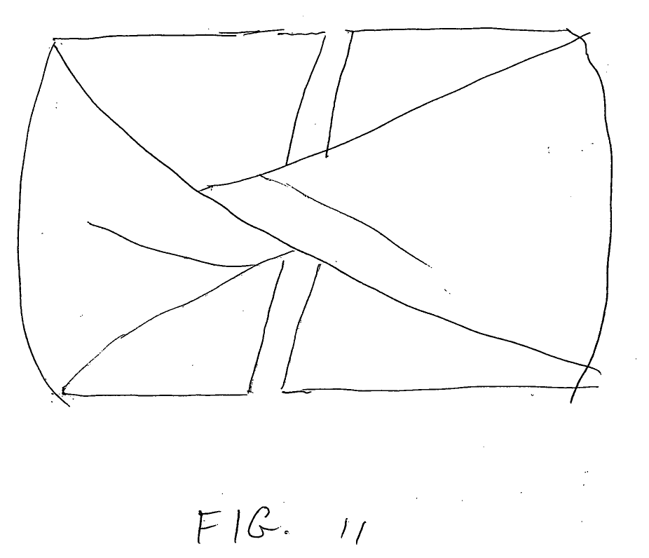

FIG. 7 is a view of the back of the invention comfortably, conveniently and securely worn as a hood. Further, the product can be positioned to serve as both hood and scarf or shawl. In this alternative manner of use the utilization of differing fabrics can be effective.

FIG. 8 is a view of yet another fashionable alternative.

FIG. 9 is a view of the back of the invention constructed by attaching the edges of a piece of cloth or fiber. The piece of material, cloth or fiber can be woven, knitted, crocheted, stitched, or manufactured in some other manner. The back can be sewn, knitted, attached, buttoned, or bonded in any other way preferred by the manufacturer and depending on intended use.

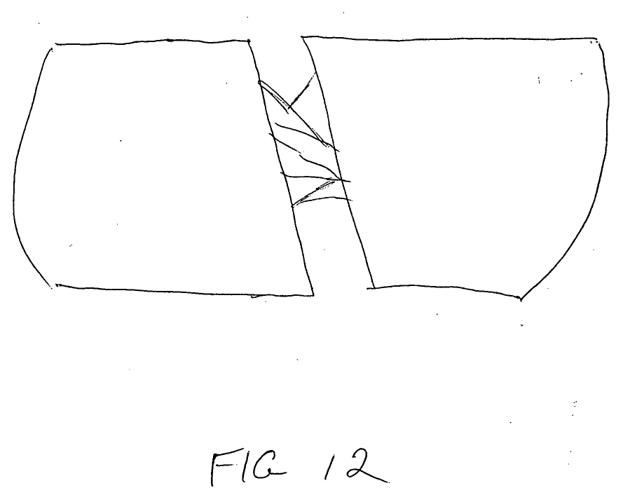

FIG. 10 is a view of a single panel prior to fold and assembly.

FIG. 11 is a view of the single panel with a fold.

FIG. 12 is a view of the invention with a fold with edges positioned prior to assembly. This illustrates the resulting and functional flexible draped neckline.

The cloth, fiber or alternative material is folded in a half twist and the back seam is secured attaching the top of the left boarder to the bottom of the right edge and the bottom of the left boarder to the top edge of the right edge. Alternatively instead of a seam, buttons or other means of fastening could be deployed.

The inventive multi-use functionality of the invention can be further enabled and enhanced by deploying a lacy knit or open weave for the material used. In such a scenario the open weave essentially causes the piece of fabric to shift accordingly when in use thereby delivering the relative same fundamental structure as the trapezoid format.

Versatility of the product can also be further enhanced by the inclusion of darts or other construction modifications.

A beneficial unique function of the invention is for sick or elderly individuals having poor circulation or other physical limitations thereby making dressing with conventional garments painful and difficult. Traditional shawls or other garments can interfere with intravenous tubing or slip into the wheels of a wheelchair. Sick and elderly individuals typically are limited to functional but often unattractive clothing. The invention eliminates the challenges of a sweater or traditional shawl and provides warmth, comfort, and secure functionality along with a fashionable appearance thereby enhancing self-esteem for the wearer.

The product can further be used as a hood in inclement weather or positioned under a coat thereby performing the function of a traditional scarf. The invention can also provide the comfort and ease of use of a shoulder blanket in a more secure manner thereby reducing the possibility of the covering item slipping into the wheels of a wheelchair onto the floor and possibly creating a safety hazard.

The product can also include a pocket. The pocket can be positioned for traditional functionality allowing the wearer the convenience of a place to hold necessary items. Further, the pocket can be positioned to provide secure positioning of a therapeutic heat sack or cool pack.

Also, the invention allows a sick or elderly person's caregiver to quickly add a layer of clothing thereby beneficially saving the caregiver's time. Additionally this scenario reduces discomfort for the patient because it eliminates the significant and repeated pain a patient can experience when their arms are manipulated into or out of sleeves.

Further, the invention can provide modesty for nursing mothers in a comfortable and convenient manner.

An alternative embodiment of the invention can include more than one type of fabric. The product consists of a first and second side, also referred to as an inner side and an outer side. The inner side and outer side can advantageously be made or constructed of mutually differing textures, fabrics, colors, or other qualities such as water repellence or absorbance. This will provide a number of advantages.

For example, one side of the shawl is made of a cozy or comfortable fabric such as cashmere or lambs wool, while the other side is of a tighter knit, even coated with a waterproof treatment such that the waterproof may be arranged as the only outer exposed fabric during rain or snow. Additionally coarse wool can be teamed with cashmere; cotton with wool. Also, embroidery, buttons, or other enhancements can be incorporated onto one or both sides of the shawl.

Further, having each side of the shawl representing one of two school colors permits the final configuration to display an attractive interweaving of the two colors.

Additionally, the upper edge of the finished shawl can be turned down to form a collar. This collar can be used to display the name of a school, other text, needlework, art, or design. The collar can allow the invention to fit more securely and warmly around the neck of a sick or elderly individual. A button, hook or other means of closure can be added to further secure the product around the neck of the wearer.

Contrasting or conflicting fabric designs may also be displayed, for example pin stripes and plaids, with the displayed design changed to match the other clothing being worn. Also, conflicting fabric designs may present a variety of unusual matches such as stripes and floral prints.

This configuration providing variants in function, design, colors, textures, warmth, water repellence, ornamentation and so forth make this product more useful, versatile and cost effective.

In use, one would first place the improved garment over the head. With the product hanging around one's shoulder or neck, the wearer positions the twist area and the body of the garment to achieve the desired use. The invention can be worn in a wide variety of ways including the following two examples:
Example (1): Twist area in front with the improved garment positioned to cover the shoulder and arms.
Example (2): The twist area positioned under the wearer's chin whereby the product is worn to cover the head.

These are, of course, mere examples of how the inventive product is best used.

Upon careful review of the foregoing specifications and drawings, it will be evident that this invention is susceptible of many modifications, combinations and alterations which might differ from those specifically set forth. Use of the term garment, shawl, shawls, poncho, scarf, scarves and hood is for the ease of description and understanding of the present concept. However, other terms with the same or similar meaning are intended to be encompassed as well, as for example muffler, drape boa, wrap, and so on.

The particular arrangements disclosed are meant to be illustrative only and not limiting as to the scope of the invention which is to be given the full breadth of claims appended hereto and any and all equivalents thereof.

What is claimed:

1. An improved garment including but not limited to:
an essentially elongated strip having two free ends wherein a 180° degree fold is given to one of the two free ends prior to applying means of attachment thereby creating an endless loop.

2. The improved garment of claim 1 in which the elongated strip is essentially a trapezoid format with the two side edges attached such that the resulting tapered shape resembles the lower portion of an inverted cone.

3. The improved garment of claim 1 in which the elongated strip is constructed of a plurality of panels.

4. The improved garment of claim 1 constructed of a material that adds warmth.

5. The improved garment of claim 1 constructed of a water repellent material.

6. The improved garment of claim 1 whose dimension is relational to the circumference of the wearer's shoulders.

7. The improved garment in claim 1 whereby the ends are attached by any of a multitude of fasteners.

8. The improved garment in claim 1 whereby two or more distinct elongated strips each with a right side and a wrong side are attached such that the wrong sides of each strip face each other with each of the right sides visible.

9. The improved garment in claim 1 with a pocket.

10. The improved garment in claim 1 whose assembly can include a pocket whereby a therapeutic hot or cold pack can be positioned securely.

11. The improved garment in claim 1 wherein said elongated strip comprises a collar portion and a shawl portion.

12. The improved garment in claim 1 wherein the front portion is secured by additional seams.

13. The improved garment in claim 1 comprising a lacy knit or open weave to enable the seeming trapezoid format thereby resulting in varying functionality.

14. An improved garment comprised of one panel or a series of panels joined together to form an essentially endless loop such that a V shaped neck, scooped neck, or otherwise draped neck results whereby varying functionality can be enjoyed by the user.

15. The improved garment of claim 14 constructed of a material that adds warmth.

16. The improved garment of claim 14 constructed of a water repellent material.

17. The improved garment in claim 14 whereby the ends are attached by any of a multitude of fasteners.

18. The improved garment in claim 14 with a pocket.

19. The improved garment in claim 14 whose assembly can include a pocket whereby a therapeutic hot or cold pack can be positioned securely.

20. The improved garment in claim 14 wherein said elongated strip comprises a collar portion and a shawl portion.
